(19) United States
(12) Patent Application Publication
(10) Pub. No.: US 2001/0031245 A1
(43) Pub. Date: Oct. 18, 2001

(54) Compositions

(75) Inventor: Roger Lidderdale Scarlett-Smith, Teddington (GB)

Correspondence Address: GLAXOSMITHKLINE CORPORATION, Corporate Intellectual Property - UW2220, P.O. Box 1539, King of Prussia, PA, 19406-0939, US

(73) Assignee: SmithKline Beecham Corporation

(21) Appl. No.: 09/780,523

(22) Filed: Feb. 9, 2001

Related U.S. Application Data
(63) Continuation of application No. 09/194,481, filed on Aug. 4, 1999, now abandoned.
Application No. 09/194,481 is a 371 of international application No. PCT/US97/09409, filed on May 30, 1997.

(30) Foreign Application Priority Data
May 31, 1996 (GB) 9611365.9

(51) Int. Cl. A61K 7/16; A61K 7/18

(52) U.S. Cl. 424/49; 424/52

(57) ABSTRACT

Oral hygiene composition are described which comprise a stannous salt and polyvinyl pyrrolidone, to counter the staining associated with use of a stannous salt.

### COMPOSITIONS

The present invention relates to oral hygiene compositions comprising a stannous salt and an anti-staining agent, for use in treating or preventing dental plaque, caries, or periodontal diseases of the oral cavity in humans or lower animals with reduced staining of teeth.

We have now surprisingly found that the development of stain may be at least substantially mitigated if not eliminated by the use of a polymeric material viz polyvinylpyrrolidone (PVP). This material is already used in some toothpastes at comparatively low levels (typically about 0.1%) as an auxiliary thickening agent and foam enhancer. In addition, it has been previously reported that PVP may be used in oral hygiene compositions, for the prevention of stains associated with the use of chlorophyll in such compositions (GB 739 936, Colgate-Palmolive Co) and also in the removal of stains, in particular tobacco tar stains, from tooth surfaces (GB 741 315, Colgate-Palmolive-Peet Co). Furthermore WO 9316681 describes the use of PVP to counter the staining associated with the use of cationic antibacterial agents such as chlorhexidine.

Accordingly, the present invention provides an oral hygiene composition comprising a bacteriostatic effective amount of a stannous salt, an anti-stain effective amount of polyvinyl pyrrolidone and an orally acceptable carrier or excipient.

Polyvinyl pyrrolidone for use in the present invention suitably has an average molecular weight in the range 5,000 to 100,000, preferably in the range 5,000 to 50,000. Polyvinyl pyrrolidones which have average molecular weights of 10,000, 30,000 and 40,000 are available from Sigma Chemjeal Co., GAF Corporation and Sigma Chemical Co. respectively. Polyvinyl pyrrolidone is suitably present in at least 1%, preferably between 2 and 30%, more preferably between 5 and 25%, and advantageously between 10 and 25% by weight of the composition.

Suitable stannous salts for use in the present invention include stannous fluoride, stannous chloride, stannous pyrophosphate and stannous chlorofluoride and mixtures thereof. Suitably, the stannous salt is present in from 0.005 to 10% preferably 0.01 to 5%, more preferably 0.01 to 2%, by weight of the oral hygiene composition.

Oral hygiene compositions of the present invention may also usefully contain an ionic fluorine-containing compound. Suitable ionic fluorine-containing compounds include, for instance, fluoride salts such as amine fluorides and alkali metal fluoride salts, for example sodium fluoride, and monofluorophosphate salts such as alkali metal monofluorophosphate salts, for example sodium monofluorophosphate. Suitably the ionic fluorine-containing compound is incorporated into the composition to provide between 100 and 3000 ppm, preferably between 500 and 2000 ppm of fluoride ions.

Oral hygiene compositions of the present invention maybe provided in any of the presentations normally used for such products, for instance, dentifrices including toothpastes and toothpowders, abrasive and non-abrasive gels, mouthwashes, gargles, irrigating solutions, mouthsprays and presentations for sucking or chewing by the user such as gums, pastilles and lozenges. Components for the orally acceptable carrier or excipient will be selected according to the particular type of presentation involved.

It will be appreciated by those skilled in the art that in order to ensure that the antibacterial efficacy of the stannous salt is not substantially diminished, compatible components will be selected for inclusion in the orally acceptable carrier or excipient.

Suitable compositions comprising a stannous salt are described in WO 9307850, WO 9403147 and WO 9509602.

Suitable nonionic surfactants include, for example, polyethoxylated sorbitol esters, in particular polyethoxylated sorbitol monoesters, for instance, PEG(40) sorbitan di-isostearate, and the products marketed under the trade name ‘Tween’ by ICI; polycondensates of ethylene oxide and propylene oxide (poloxamers), for instance the products marketed under the trade name ‘Pluronic’ by BASF-Wyandotte; condensates of propylene glycol; polyethoxylated hydrogenated castor oil, for instance, cremophors; and sorbitan fatty esters.

Suitable amphoteric surfactants include, for example, long chain imidazoline derivatives such as the product marketed under the trade name ‘Miranol C2M’ by Miranol; long chain alkyl betaines, such as the product marketed under the tradename ‘Empigen BB’ by Albright+Wilson, and long chain alkyl amidoalkyl betaines, such as cocamidopropylbetaine, and mixtures thereof.

Suitable cationic surfactants include the D,L-2-pyrrolidone-5-carboxylic acid salt of ethyl-N-cocoyl-L-arginate, marketed under the trade name CAE by Ajinomoto Co. Inc.

Advantageously, the surfactant is present in the range 0.005 to 20%, preferably 0.1 to 10%, more preferably 0.1 to 5% by weight of the dentifrice.

Suitable nonionic thickening agents include, for example, (C₁₋₆)-alkylcellulose ethers, for instance methylcellulose, hydroxy(C₁₋₆)-alkylcellulose ethers, for instance hydroxypropylcellulose, (C₂₋₆)-alkylene oxide modified (C₁₋₆)-alkylcellulose ethers, for instance hydroxypropyl methylcellulose, and mixtures thereof. Advantageously the nonionic thickening agent is present in the range 0.01 to 30%, preferably 0.1 to 15%, more preferbly 1 to 5%, by weight of the composition.

Suitable sparingly soluble salts that may be used as an abrasive include calcium carbonate, calcium phosphates, magnesium carbonate, insoluble sodium metaphosphate, and suitable mixtures thereof. The agent to suppress anion formation typically comprises a water soluble salt containing a cation which may be same as the cation of the abrasive and which forms an essentially insoluble or sparingly soluble salt with the anion of the abrasive. Preferably the sparingly soluble salt used as an abrasive is calcium carbonate, advantageously used in combination with dicalcium phosphate, which also usefully buffers the pH of the formulation. Suitable types of calcium carbonate include both natural and synthetic chalks. The agent to suppress anion formation may be an alkaline earth metal salt, for instance calcium chloride. The agent is preferably present in from 0.0001 to 1%, more preferably 0.005 to 0.1% by weight of the dentifrice.

The term ‘essentially insoluble compound’ as used herein refers to a compound which is intrinsically insoluble in aqueous solution and includes those compounds which are listed as being ‘insoluble’ in cold water in the ‘Handbook of Chemistry and Physics’, 48th Edition, Chemical Rubber Company, Section B, Physical Constants of Inorganic Compounds. Furthermore, such compounds when used as an abrasive shall contain little if any contaminating anionic impurities. Preferably the insoluble abrasive compound should contains less than 1%, preferably less than 0.5%, and more preferably less than 0.25% of anionic impurities, based on the weight of the abrasive. Suitable essentially insoluble compounds for use as abrasives include, for example, silica, zinc orthophosphate, plastics particles, alumina, hydrated alumina, and calcium pyrophosphate or mixtures thereof.

Preferably, the abrasive is silica. Suitable silicas include natural amorphous silica, for instance diatomaceous earth; and synthetic amorphous silicas, for instance a precipitated silica, or a silica gel, such as a silica xerogel; or mixtures thereof. The preferred synthetic amorphous silicas are those with a low-level of water soluble anionic impurities (hereinafter referred to as “low-anion silica”). These are obtainable from manufacturing process which are carefully controlled so that the level of anion impurities, particularly sulphate and silicate from sodium sulphate and sodium silicate, respectively, is kept to a minimum. Alternatively, or in addition, the level of anion impurities may be reduced to the required level by careful washing of the initially produced silica with, for instance, deionised or distilled water. Suitable low-anion silicas contain less than 0.5%, preferably less than 0.25%, more preferably less than 0.1% by weight of water soluble impurities such as sodium sulphate and/or sodium silicate. Examples of such low-anion silicas are described in EP A 0 315 503 (to Rhone-Poulenc). Suitable silica xerogels are described in U.S. Pat. No. 3,538,230.

Preferred precipitated silicas include the grade RP93 available from Rhone-Poulenc and those marketed under the trade name ‘SIDENT’ by Degussa, for instance, SIDENT 9 silica. Preferred silica xerogels are those marketed under the trade name ‘SYLOBLANC’ by W. R. Grace Corporation, Davison Chemical Division. Suitable grades of precipitated silica have BET surface areas in the range 20 to 300, preferably 20 to 100 m²/g and median agglomerate sizes in the range 2 to 50, preferably 5 to 30 μm. Suitable forms of diatomaceous earth include those marketed under the trade name ‘Celite’ by Johns-Manville Products Corporation, for instance ‘Celite Superfine Superfloss’.

The abrasive is advantageously present in the range 1 to 80%, preferably 5 to 70%, more preferably 5 to60% by weight of the dentifrice.

It will be appreciated that each of the thickening agent, the surfactant and abrasive should be, at the level employed in the dentifrice, compatible with the stannous salt, that is, each will not substantially reduce the availability of the stannous salt, for instance, by more than 30%, preferably more than 20%. This may be confirmed by, for instance, determining the biological activity of the formulation, by conventional microbiological assay using, for instance, M. luteus as the assay organism in a standard agar diffusion method, in the presence and absence of each of the aforementioned thickening agent, surfactant and abrasive.

Mouthwashes according to the present invention will preferably comprise as components of the carrier a surfactant and a humectant in an aqueous or an aqueous/ethanol solution. Gels according to the present invention will preferably comprise as components of the carrier a surfactant, humectant, thickening agent and optionally water. Dentifrices according to the present invention will preferably comprise as components of the carrier a surfactant, humectant, thickening agent, abrasive and if necessary, water.

Suitable humectants for use in compositions of the invention include for instance glycerine, sorbitol, propylene glycol or polyethylene glycol, or mixtures thereof The humectant may be present in the range from 5 to 70%, preferably 5 to 30%, more preferably 10 to 30% by weight of the dentifrice.

Other materials may be added to the compositions if required, for instance sweetening agents, flavouring agents, colouring and whitening agents, preservatives and emulsifiers.

The pH of a composition according to the invention will be orally acceptable and typically in the range pH 5 to 9.

Oral hygiene compositions according to the present invention may be prepared by mixing the ingredients thereof in the required proportions and in any order that is convenient and thereafter and if necessary adjusting the pH to the required value.

Oral hygiene compositions according to the present invention are of use in reducing or eliminating the stain normally asociated with the use of a bacteriostatic stannous salt. Accordingly, in a further aspect, the present invention provides for an oral hygiene composition as hereinbefore defined for use in therapy, in particular anti-plaque, anti-caries, anti-calculus and/or periodontal (including anti-gingivitis) therapy. The present invention also provides for the use of polyvinyl pyrrolidone and a stannous salt in the manufacture of an oral hygiene composition for use in oral hygiene.

In oral hygiene compositions according to the present invention, the stannous salt and the polyvinyl pyrrolidone anti-stain agent will normally be incorporated together in a single oral hygiene composition. The stannous salt and the polyvinyl pyrrolidone anti-stain agent may however be also provided in separate oral hygiene compositions which may be used separately, simultaneously or sequentially. Accordingly, in a further aspect, the present invention provides an oral hygiene kit comprising as a first item an oral hygiene composition comprising an anti-stain effective amount of polyvinyl pyrrolidone and an orally acceptable excipient or carrier and, as a separate second item, an oral hygiene composition comprising a bacteriostatic effective amount of a stannous salt and an orally acceptable excipient or carrier. It will be appreciated that in such an oral hygiene kit, the two oral hygiene compositions may be of the same or different character. Thus, for instance, the stannous salt may be provided in a dentifrice whilst polyvinylpyrrolidone is provided in a mouthwash or gargle or vice versa. Alternatively both may be provided as, for example, a dentifrice, mouthwash or gargle.

In a further aspect, the present application further provides an oral hygiene composition comprising an antistain effective amount of polyvinyl pyrrolidone and an orally acceptable excipient or carrier.

The invention will now be illustrated by reference to the following examples.

### EXAMPLE 1—Efficacy of PVP in a stain model

The efficacy of polyvinylpyrrolidone (PVP) in preventing the formation of stain associated with the use of stannous fluoride was evaluated in vitro in a model system which consisted of exposing sintered hydroxyapatite (HA) discs to a tea solution and comparing the stain formation when treating them with stannous fluoride in the absence or presence of polyvinylpyrrolidone.

The colour of a HA disc was measured using a chromameter to establish a baseline figure. The disc was then rinsed with deionised water for 1 min and incubated in pooled filtered human saliva for 2 hour at 37° C., to induce pellicle formation. After further rinsing for 1 min with deionised water, the disc was immersed for 2 min in a solution containing stannous fluoride (0.4% w/v)), after which the disc was rinsed with deionised water (1 minute) and then immersed in cold tea for 3 hr. The disc was then rinsed with deionised water for 1 min, immersed in pooled filtered human saliva for 1 hr at 37° C., rinsed with deionised water for 1 min, subjected to a 2 min application of stannous fluoride (0.4% w/v), rinsed with deionised water for 1 min and then immersed in cold tea overnight. The procedure was repeated daily for 5 days. At the end of the 5 days, the colour of the disc was determined using the chromameter. The degree of colour change was calculated using the formula:

E*ab=ν[ΔL²+Δa²+Δb²]

in which E*ab is colour difference

L is degree of black/white colour

a is degree of green/red colour

b is degree of blue/yellow colour.

In each treatment group, 4 discs were used.

The procedure was then repeated, but using a solution containing stannous fluoride (0.4% w/v) PVP (av. mol. wt. 40,000, at 1, 5, 10 and 20%) instead of stannous fluoride alone. In addition, a control was run in which the disc was treated with neither stannous fluoride or PVP. Results obtained demonstrated that PVP reduced 0.4% stannous fluoride induced stain formation on hydroxyapatite discs in a concentration dependent manner between 1% and 20% PVP. Stain was significantly reduced (P<0.05) by 27.4% and 24.5% for 10% and 20% PVP respectively.

EXAMPLE 2
toothpaste

| | |
|---|---|
| Glycerine | 8.0% |
| Hydroxypropyl methylcellulose^{a} | 3.40 |
| Polyvinyl pyrrolidone^{b} | 20.00 |
| Stannous fluoride | 1.0 |
| Sodium saccharin | 0.10 |
| Silica^{c} | 16.00 |
| Polyoxyethylene-polyoxypropylene block copolymer^{d} | 2.00 |
| Sodium fluoride | 0.22 |
| Talin | 0.02 |
| Flavour | 1.00 |
| Deionised water | qs |

^{a}Methocel K15M premium and Methocel K100LV premium in ratio 1:4.
^{b}average molecular weight 30,000
^{c}P293 from Rhone-Poulene
^{d}Pluronic F108 (BASE-Wyandolte).

EXAMPLE 3
toothpaste

| | |
|---|---|
| Glycerine | 10.00% |
| 70% sorbitol sol'n | 20.00 |
| Stannous chloride | 1.0-1.5 |
| Stannous fluoride | 0.454 |
| Saccharin | 0.40 |
| Titanium dioxide | 1.00 |
| Sodium hydroxide | 0.20 |
| Amorphous silica | 20.00 |
| Sodium lauryl sulphate | 1.50 |
| Carboxymethyl cellulose | 0.70 |
| PVP | 20.00 |
| Flavour | 1.00 |
| Deionised water | qs |

1. An oral hygiene composition comprising a bacteriostatic effective amount of a stannous salt, an anti-stain effective amount of polyvinyl pyrrolidone and an orally acceptable carrier or excipient.

2. An oral hygiene composition as claimed in claim 1 in which polyvinyl pyrrolidone has an average molecular weight in the range 5,000 to 100,000.

3. An oral hygiene composition as claimed in claim 1 or 2 in which polyvinyl pyrrolidone has an average molecular weight in the range 5,000 to 50,000.

4. An oral hygiene composition as claimed in any one of claims 1 to 3 in which polyvinyl pyrrolidone is present in from at least 1% by weight of the composition.

5. An oral hygiene composition as claimed in any one of claims 1 to 4 in which polyvinyl pyrrolidone is present in from 2 to 30% by weight of the composition.

6. An oral hygiene composition as claimed in any one of claims 1 to 5 in which polyvinyl pyrrolidone is present in from 5 to 25% by weight of the composition.

7. An oral hygiene composition as claimed in any one of claims 1 to 6 in which polyvinyl pyrrolidone is present in from 10 to 25% by weight of the composition.

8. An oral hygiene composition as claimed in any one of claims 1 to 7 in which the stannous salt is selected from the group consisting of stannous fluoride, stannous chloride, stannous pyrophosphate and stannous chlorofluoride and mixtures thereof

9. An oral hygiene composition as claimed in claim 8 in which the stannous salt is stannous fluoride.

10. An oral hygiene composition as claimed in any one of claims 1 to 9 further comprising an ionic fluorine-containing compound.

11. An oral hygiene composition as claimed in any one of claims 1 to 10 which is a mouthwash.

12. An oral hygiene composition as claimed in any one of claims 1 to 10 which is a dentrifice in which the surfactant, thickening agent and abrasive are selected for compatability with the stannous salt.

13. An oral hygiene composition as claimed in claims 12 in which the dentifrice comprises a non-ionic, cationic or amphoteric surfactant, or a mixture thereof; a non-ionic thicnening agent and an abrasive which is either an essentially insoluble compound and substantially devoid of water soluble anionic impurities or a sparingly soluble compound used in conjunction with an agent to suppress anion formation, or a mixture thereof.

14. An oral hygiene composition as defined in claim 13 in which the surfactant comprises a non-ionic surfactant.

15. An oral hygiene composition as claimed in claim 13 or 14 in which the abrasive is a low-anion silica abrasive.

16. An oral hygiene composition as claimed in claim 15 in which the silica abrasive comprises less than 0.5% by weight of sodium sulphate.

17. An oral hygiene composition as defined in any one of the preceding claims for use in therapy.

18. A method of treating plaque, caries, calculus, gingivitis and/or periodontal disease with a cationic anti-bacterial agent with reduced staining which method comprises treating the patient in need thereof with an effective amount of a composition as defined in any one of claims 1 to 17.

19. The use of polyvinyl pyrrolidone and a stannous salt in the manufacture of a composition for use in oral hygiene with reduced staining.

20. An oral hygiene kit comprising as a first item an oral hygiene composition comprising an anti-stain effective amount of polyvinyl pyrrolidone and an orally acceptable carrier or excipient and, as a separate second item, an oral hygiene composition comprising a bacteriostatic effective amount of a stannous salt and an orally acceptable carrier or excipient.

21. An oral hygiene kit as claimed in claim 20 for use in therapy.

22. A method of reducing or eliminating the stain associated with the use of a stannous salt in oral hygiene which method comprises administering to a patient in need thereof an oral hygiene composition comprising an anti-stain effective amount of polyvinyl pyrrolidone separately, simultaneously or sequentially with an oral hygiene composition comprising a bacteriostatic effective amount of a stannous salt.

23. The use of polyvinyl pyrrolidone in the manufacture of an oral hygiene composition for use, separately, simultaneously or sequentially, with an oral hygiene composition comprising a bacteriostatic effective amount of a stannous salt, to reduce or eliminate the stain associated with the use of an oral hygiene composition comprising a stannous salt.

24. A process for preparing an oral hygiene composition as defined in any one of claims 1 to 16 which process comprises admixing the ingredients in the appropriate amounts in any order that is convenient and, if necessary, adjusting the pH to the final required value.
